(19) United States
(12) Patent Application Publication
(10) Pub. No.: US 2020/0337887 A1
(43) Pub. Date: Oct. 29, 2020

(54) CONDOM WITH RING THAT CONNECTS TO THE BACK

(71) Applicant: Kevin Jackson, Suffield, CT (US)

(72) Inventor: Kevin Jackson, Suffield, CT (US)

(21) Appl. No.: 16/873,497

(22) Filed: Apr. 22, 2020

Related U.S. Application Data
(60) Provisional application No. 63/100,612, filed on Mar. 23, 2020.

(51) Int. Cl. A61F 6/04

(52) U.S. Cl. CPC A61F 6/04; A61F 2006/047

(57) ABSTRACT

A condom with a firm rubber cirular washer shaped ring with a wider perimeter then the condom that can connect and disconnect to the back of the condom. The ring is made to push against the exterior of the vagina during sex and cause the condom to stay on the penis.

### FIELD

The present application relates to a condom with a round washer shaped ring that connects to the back of it.

### BACKROUND

Regular condoms provide protection from sexually transmitted diseases and provide birth control but they also reduce pleasure by blocking the direct contact of the genitals. They also cannot be applied with a generous amount of lubercation on the inside of them without the risk of them sliding off increasing. Furthermore, the decreasing of the penis size upon ejaculation, or semen can cause the condom to slip off during sex.

### SUMMARY

By having a condom with a round washer shaped rubber ring that connects to the back of the condom and has a wider perimeter then the condom, it allows a generous amount of lubercation to be applied to the inside of the condom or to the penis before the condom is put on and it eliminates the risk of it sliding off or getting stuck in the womens vagina during sex.

With the washer shaped rubber ring sticking out about an inch to a half an inch around the back of the condom, everytime the penis pushes foward, the ring would push against the exterior of the vaginas outer area and cause the condom to stay on the penis regardless of how moist or wet the inside of it is.

The increased moisture inside the condom will provide more pleasure for both the male and female and create a safer sexual encounter.

### BRIEF DESCRIPTION

In the following detailed portion of the present description the teachings of the present application will be explained in more detail with references to example embodiments shown in the drawings, in which:

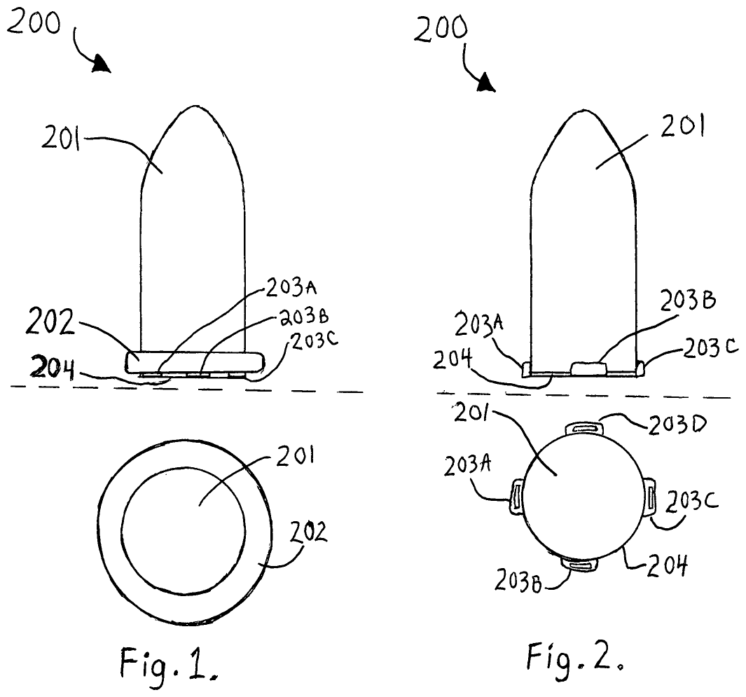

FIG. 1 is an isometric view of a condom with a washer shaped ring according to a first embodiment.

FIG. 2 is an isometric view of a condom with a washer shaped ring that connects to it but without the washer shaped ring connected according to a second embodiment.

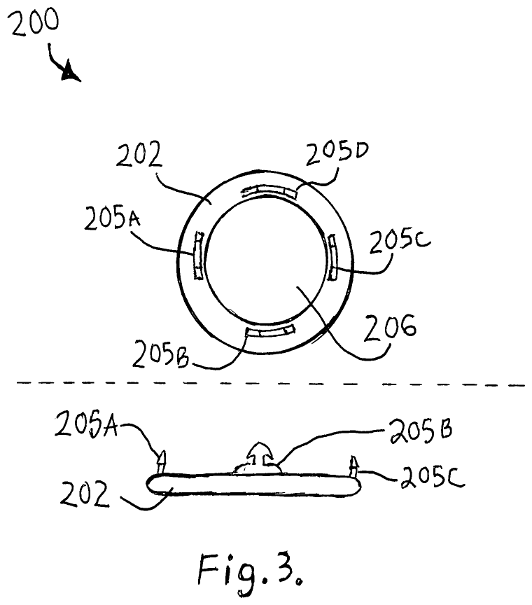

FIG. 3 is an isometric view of a washer shaped ring that connects to a condom but without the condom according to a third embodiment.

### DETAILED DESCRIPTION

In the following detailed description, the condom with a washer shaped ring attached according to the teachings for this application in the form of a condom will be described by the embodiments. It should be noted that although it is described as a ring that can be removed, the teachings of this application can also be used for a ring that is permanently attached. The purpose of it being removable is because it would be difficult to pack a condom with a ring attached and so the user can have the option of using the ring or not.

The first embodiment of the condom 200 is illustrated in FIG. 1. The condom 200 comprises a housing 201 with a firm rubber washer shaped ring 202 that connects to the condom.

The body of the condom 201 is the thin rubber portion of the condom that fits over the penis and is the shape of a penis. The top round portion of the condom 201 is the tip of the condom that fits over the tip of the penis. There is a firm rubber washer shaped ring 202 shown in the present embodiment which is shown connected to the condom 201 but it can disconnect. First the condom 201 is rolled on to the penis alone. Then, after the condom 201 is on, the rubber ring 202 is slid on the condom threw its middle which is hollow and there are hooks on the ring 202 which are later shown in FIG. 3, that connect with four insertion grooves 203A, 203B, 203C, 203D, that are on the condoms back end 204. When the ring 202 is connected to the four insertion grooves 203A, 203B, 203C, 203D, the ring snaps in place and cannot move. The four grooves 203A, 203B, 203C, 203D, are made of hard plastic but cannot be fully seen in this embodiment because the ring 202 covers them once it is on the condom. 201. Between the four grooves 203A, 203B, 203C, 203D, is the condoms back end 204 which is made of rubber a little thicker then the condoms body 201 and is no different then the back end of a regular condom and is a rubber that is able the stretch.

The ring 202 has a hollow inside that allows the condom to go threw the middle of it. It is made of a circular firm rubber and has a perimeter that is wider then the condom. Its purpose is to connect to the back of the condom and for it to push against the exterior of the vagina and thereby force the condom to stay in place and be unable to slide off during sex.

A second embodiment of the condom is illustrated in FIG. 2. The condom 200 according to the second embodiment is an isometrical view of the condom without the ring 202 attached. At the condoms back end 204, there are four plastic insert holes 203A, 203B, 203C, 203D, which four hooks later shown in FIG. 3. fit into. The insert grooves are tiny rectangular shaped platic boxes that are glued or attached to the condom.

A third embodiment of the condom is illustrated in FIG. 3. The condom according to the third embodiment is an isometrical view of the ring which connects to the condom 201 but is shown without the condom attached. The rings center 206 is a hollow circular hole and is where the condom goes threw after the condom is on the penis. On the ring 202 there are four hooks 205A, 205B, 205C, 205D, that insert into the four insert grooves or holes 203A, 203B, 203C, 203D, shown in FIG. 2. and snap into place, which are also made of hard plastic and are built into the rubber ring 202.

The teachings of this application are described in detail for the purpose of illustration, it is to be understood that such detail is soley for that purpose, and variations can be made by those skilled in the art without departing from the scope of the teaching.

The teachings of the present application is presented as a rind that attaches and detaches from a condom. It is to be understood that the ring can be made to stay on the condom permanently and be made to not be removed. It is to be noted that althought this application is used so that the penis can be lubercated before the condom is put on, it is also so the sex is safer even if there is no lubercation applied. It is also to be noted that features can be added to the ring such as a vibrator or anything else that would enhance the sexual experiance. Features described in the preceeding description may be used in combination other then the combinations explicitly describe.

Whilst endeavouring in the foregoing specification to draw attention to these features of the invention believed to be of particular importance, it should be understood that the Applicant claims protection in respect of any patentable features or combination of features hereinbefor reffered to and/or shown in the drawings whether or not particular emphasis has been placed thereon.

The term “comprising” as used in the claims does not exclude other elements or steps. The term “a” or “an” in the claims does not exclude a plurality. A unit or other means may fulfill the functions of several units or means recited in the claims. The term “ring” used in the claims means a circular flat washer shaped rubber piece of the device. The term “washer shaped” used in the claims means the shape of a washer used with a screw. The term “grooves” used in the claims means a small hole that a hook can be inserted into.

1. A male condom with a back end that has a perimeter or diameter that is wider then the perimeter of the condom.

2. A male condom with an attachable and detachable back end that has a perimeter or diameter that is wider then the perimeter of the condom.

3. A male condom with a circular washer shaped back end that has a perimeter that is wider then the perimeter of the condom.
